(19) United States
(12) Patent Application Publication
(10) Pub. No.: US 2009/0142797 A1
(43) Pub. Date: Jun. 4, 2009

(54) BLOOD DILUENT AND METHOD OF USE THEREOF

(75) Inventors: Lina Zhang, Shenzhen (CN); Dan Liu, Shenzhen (CN); Wenjuan Xu, Shenzhen (CN); Mulong Liu, Shenzhen (CN)

Correspondence Address: MINDRAY C/O STOEL RIVES LLP, 201 S. MAIN STREET, SUITE 1100, SALT LAKE CITY, UT, 84111, US

(73) Assignee: SHENZHEN MINDRAY BIO-MEDICAL ELECTRONICS CO., LTD., Shenzhen (CN)

(21) Appl. No.: 11/966,713

(22) Filed: Dec. 28, 2007

(30) Foreign Application Priority Data
Dec. 4, 2007 (CN) 200710199435.3

(51) Int. Cl. C07D 487/04; C12Q 1/00

(52) U.S. Cl. 435/40.5; 544/264; 544/265; 544/267; 544/274; 544/273

(57) ABSTRACT

Blood diluent for use in the analysis of blood components may include at least one purine compound or salt thereof. The blood diluent may also include at least one of alkali metal salt and/or at least one organic buffers and/or inorganic buffer. The blood diluent may be utilized to stabilize blood cells during storage and facilitate the analysis thereof.

### RELATED APPLICATIONS

This application claims priority to Chinese Patent Application No. 200710199435.3, filed Dec. 4, 2007, which is incorporated herein by reference in its entirety.

### TECHNICAL FIELD

The present disclosure relates to a blood diluent and method of use thereof, and more particularly to a blood diluent for use in blood cell analyzers and method of use thereof.

### SUMMARY

A blood diluent for use in the analysis of blood components is disclosed. In one embodiment, the blood diluent comprises one or more purine compounds or salts thereof and/or at least one or more alkali metal salts and/or at least one or more organic buffers and/or inorganic buffers.

### BRIEF DESCRIPTION OF THE DRAWINGS

FIG. 1 is a histogram illustrating distribution of white blood cell subpopulations obtained by a blood analyzer.

FIGS. 2-4 are each a diagram of blood sample analysis using one embodiment of the blood diluent of the present disclosure containing 0.05 g/L, 0.5 g/L and 25 g/L of caffeine respectively.

FIGS. 5-6 are each a diagram of blood sample analysis using one embodiment of the blood diluent of the present disclosure containing caffeine, at pH 5.0 and 9.5 respectively.

FIG. 7 illustrates a comparison of a blood sample analysis using one embodiment of the blood diluent of the present disclosure containing aminophylline (right) with a blood sample analysis without using a blood diluent of the present disclosure (left).

FIG. 8 illustrates comparison of a blood sample analysis using one embodiment of the blood diluent of the present disclosure containing theophylline (right) with a blood sample analysis without using a blood diluent of the present disclosure (left).

FIG. 9 illustrates comparison of a blood sample analysis using another embodiment of the blood diluent of the present disclosure containing uric acid (right) with a blood sample analysis without using a blood diluent of the present disclosure (left).

FIG. 10 illustrates comparison of a blood sample analysis using one embodiment of the blood diluent of the present disclosure containing caffeine (right) with a blood sample analysis without using a blood diluent of the present disclosure (left).

FIG. 11 illustrates comparison of a blood sample analysis using yet another embodiment of the blood diluent of the present disclosure containing hypoxanthine (right) with a blood sample analysis without using a blood diluent of the present disclosure (left).

### DETAILED DESCRIPTION

In the blood and hemopoietic organs of normal human beings, the number of each kind of blood cell may be in a certain normal range, and each different kind of blood cell and each different stage of cell development have certain characteristics in morphology and structure. Routine blood examinations involve such parameters as hemoglobin (HGB) concentration, red blood cell count (RBC), white blood cell count (WBC), platelet (PLT) count, hematocrit (HCT) level, mean cell volume (MCV), mean cell (erythrocyte) hemoglobin (MCH), mean cell (erythrocyte) hemoglobin concentration (MCHC).

Presently, many instruments produce cell data results along with diagrams of cell volume distribution representing the distribution of cell populations in a sample. Such diagrams are often referred to as cell distribution histograms.

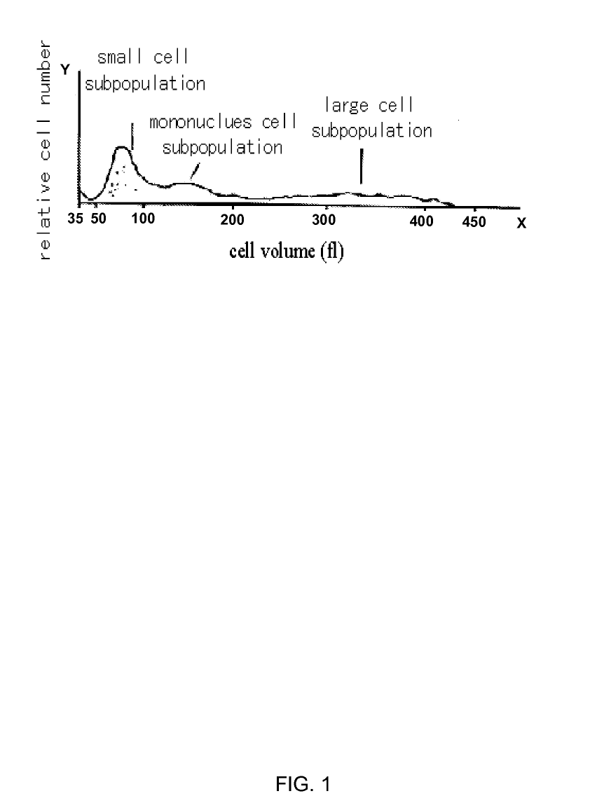

Referring to FIG. 1, white blood cells treated with a lysing agent (hemolytic agent) may be differentiated into the following cell subpopulations: 1) small-cell subpopulation, which is primarily lymphocytes; 2) mononuclear-cell subpopulation, or middle cell subpopulation (MID), which includes monocytes, eosinophils, basophils, juvenile cells, and leukemia cells (if present); and 3) a large-cell subpopulation, which may primarily include neutrophils (GRAN). The percentage of each subpopulation is calculated by the instrument according to the ratio of each subpopulation relative to the total number of cells. The absolute number of each subpopulation is obtained by multiplying the respective percentage by the total number of white blood cells in the sample. Therefore, the shape of the white blood cell histogram can be an important reference for clinical diagnosis.

As routine blood examinations in foreign medical institutions mostly employ anticoagulant venous blood as the sample, foreign manufacturers of blood cell analyzers and reagents often focus on the accuracy of counting and differentiation in the whole-blood mode, with special consideration of such characteristics as osmotic pressure and conductivity of the reagents. However, in the domestic practice, capillary blood is often collected as the sample when routine blood examinations are conducted. Many domestic medical institutions store the capillary blood for a long time after collection. However, improper storage of blood without a proper blood diluent, may result in unstable and inaccurate white blood cell histograms which may lead to the collection of erroneous data and to difficulties in clinical diagnosis.

The present disclosure provides a blood diluent, which allows prolonged storage of capillary blood and accurate and stable counting and determination of such indexes for blood analysis as white blood cells, red blood cells, platelets and hemoglobin both in the whole blood mode and in the pre-dilution mode, and meanwhile ensures that the characteristics of white blood cell subpopulations would not be changed.

In one embodiment, the present disclosure provides a blood diluent for use in the analysis of blood components, said blood diluent comprising one or more purine compounds or salts thereof. Said blood diluent optionally further comprises a) one or more alkali metal salts; and/or b) one or more organic buffers and/or inorganic buffers.

In another embodiment, the present disclosure provides a method for analyzing blood components, said method comprising the step of mixing a blood sample with one embodiment of a blood diluent of the present disclosure and an optional lysing agent such as a hemolytic agent.

It is has been clinically shown that the present disclosure can provide an accurate determination of various routine blood parameters and maintain the stability of white blood cell differentiation for more than 2 hours after the collection of capillary blood. Moreover, the blood samples stabilized with the blood diluent of the present disclosure are suitable for analysis of blood components in various kinds of blood cell analyzers under various detection conditions.

One embodiment of the blood diluent of the present disclosure is used for diluting blood samples to provide a suitable environment of conductivity and osmotic pressure for methods of cell detection that rely on electrical impedance to stabilize various blood cells and for distinguishing their morphology. For example, one embodiment of the blood diluent of the present disclosure may be utilized to stabilize white blood cells to facilitate their separation into various subpopulations based on electrical impedance, thereby achieving the counting of white blood cell subpopulations.

“Purine compounds” as used herein refers to compounds having the structure of pyrimidoimidazole fused ring system as shown below and derivatives thereof.

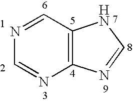

The amino and hydroxy derivatives of purine are widely present in animals and plants. Important drugs such as theobromine, caffeine and theophylline may include purine compounds. The components of nucleic acids and metabolic products thereof such as xanthine, hypoxanthine and uric acid, also include all purine compounds.

More particularly, the structures of hypoxanthine, xanthine, caffeine, theophylline (theosine), uric acid, diprophylline (dyphylline) and theobromine are as follows:

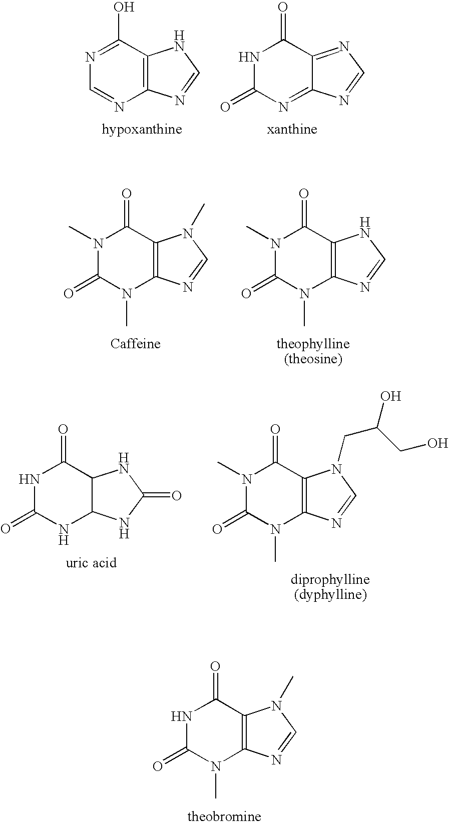

According to the present disclosure, the stabilization of blood components such as white blood cells, red blood cells, platelets and hemoglobin etc., especially white blood cells, may be accomplished through the use of one or more purine compounds.

Purine compounds or salts thereof for use in various embodiments of the blood diluent of the present disclosure, may include hypoxanthine or xanthine compounds or salts thereof. Other embodiments of a blood diluent according to the present disclosure may include, but are not limited to, the following substances and derivatives and salts thereof:

1. caffeine and derivatives thereof;

2. theophylline and derivatives thereof, for example theophylline, aminophylline, choline theophyllinate, diprophylline, doxofylline, cafedrine, bamifylline, theobromine, pentoxifylline, propentofylline, denbufyline etc.;

3. hypoxanthine or xanthine and derivatives thereof, for example hypoxanthine, xanthine, 1-methylxanthine, 3-methylxanthine, 7-methylxanthine, 1,7-dimethylxanthine etc.; and/or

4. uric acid and derivatives thereof, for example 1-methyluric acid, 1,3-dimethyluric acid, 7-methyluric acid etc.

Those skilled in the art will recognize that the derivatives and salts of the one or more purine compounds may include, but are not limited to, salts formed with an acid, salts formed with a base, esters, N-oxides, tautomers, solvates, complexes, other substances formed with other radicals through covalent or noncovalent bonding and precursors or metabolic products thereof.

In one embodiment, one or more purine compounds may be present in the blood diluent in an amount ranging from approximately 0.05 g/L to approximately 25 g/L. In another embodiment, one or more purine compounds may be present in the blood diluent in an amount ranging from approximately 0.3 g/L to approximately 10 g/L.

In one embodiment, one or more alkali metal salts may be used for providing a suitable conductivity and osmotic pressure for a blood diluent of the present disclosure. In another embodiment, one or more alkali metal salts may be selected from alkali metal sulfates, alkali metal halides and other alkali metal salts, including sodium sulfate and sodium chloride. In one embodiment of a method of blood cell analysis, a blood diluent may include conductivity in the range of approximately 16 mS/cm (milliSiemens per centimetre) to approximately 22 mS/cm. The conductivity may be identified, for example, by an instrument sold as METTLER 326. In one embodiment of a method of blood cell analysis, a blood diluent may include osmotic pressure in the range of approximately 250 mOsm/Kg (milliosmoles per kilogram) to approximately 350 mOsm/Kg. The osmotic pressure may be identified, for example, by an instrument sold as OSMOMETER: SLAMED 800 cl.

One embodiment of the blood diluent of the present disclosure may contain buffers for adjusting the pH value of the blood diluent. The buffers may be those buffers commonly used in the art including common buffering systems such as formic acid, phthalic acid, acetic acid, phosphoric acid, TRIS, boric acid, carbonic acid and salts thereof. In one embodiment, the pH range of the blood diluent of the present disclosure can be adjusted in the range of approximately pH 5.0-9.5.

The present disclosure also provides methods for analyzing a blood sample or blood components, said method comprising mixing the blood sample with a blood diluent, according to the various embodiments of the present disclosure. Embodiments of the methods for analyzing a blood sample or blood components may include mixing the blood sample with one or more lysing agents or hemolytic agents and determining the parameters of various components in the blood such as white blood cells, red blood cells, platelets, hemoglobin.

It is preferable to use a lysing agent, such as a hemolytic agent, to separate the red blood cells from the white blood cells during the blood sample analysis.

### EXAMPLES

The various embodiments of a blood diluent according to the present disclosure are further described by way of the following examples. However, it is understood that the present disclosure is not limited thereto or thereby.

In the following examples of preparation of the embodiments of a blood diluent of the present disclosure, the blood diluent may be obtained by mixing the components as listed.

### Example 1

In this example, a blood diluent is made by mixing the following into a final volume of 1 L:

| | | |
|---|---|---|
| caffeine | 0.05 | g |
| sodium sulfate | 10 | g |
| sodium chloride | 3.5 | g |
| phosphate buffer solution | 0.05 | M |
| add water to make | 1 | L |
| pH 7.0 | | |

### Example 2

In this example, a blood diluent is made by mixing the following into a final volume of 1 L:

| | | |
|---|---|---|
| caffeine | 0.5 | g |
| sodium sulfate | 10 | g |
| sodium chloride | 3.5 | g |
| phosphate buffer solution | 0.05 | M |
| add water to make | 1 | L |
| pH 7.0 | | |

### Example 3

In this example, a blood diluent is made by mixing the following into a final volume of 1 L:

| | | |
|---|---|---|
| caffeine | 25 | g |
| sodium sulfate | 10 | g |
| sodium chloride | 3.5 | g |
| phosphate buffer solution | 0.05 | M |
| add water to make | 1 | L |
| pH 7.0 | | |

### Example 4

In this example, a blood diluent is made by mixing the following into a final volume of 1 L:

| | | |
|---|---|---|
| caffeine | 0.5 | g |
| sodium sulfate | 10 | g |
| sodium chloride | 3.5 | g |
| phosphate buffer solution | 0.05 | M |
| add water to make a total volume of | 1 | L |
| pH 5.0 | | |

### Example 5

In this example, a blood diluent is made by mixing the following into a final volume of 1 L:

| | | |
|---|---|---|
| caffeine | 0.5 | g |
| sodium sulfate | 10 | g |
| sodium chloride | 3.5 | g |
| phosphate buffer solution | 0.05 | M |
| add water to make | 1 | L |
| pH 9.5 | | |

### Example 6

In this example, a blood diluent is made by mixing the following into a final volume of 1 L:

| | | |
|---|---|---|
| aminophylline | 0.5 | g |
| sodium sulfate | 10 | g |
| sodium chloride | 3.5 | g |
| phosphate buffer solution | 0.05 | M |
| add water to make | 1 | L |
| pH 7.0 | | |

### Comparative Example 1

In this example, a blood diluent is made by mixing the following into a final volume of 1 L:

| | | |
|---|---|---|
| N-(2-acetamido)-2-iminodiacetic acid (ADA) | 0.1 | g |
| dimethylurea | 0.5 | g |
| (2-pyridinethiol-1-oxide) sodium | 0.1 | g |
| sodium sulfate | 10 | g |
| sodium chloride | 3.5 | g |
| phosphate buffer solution | 0.05 | M |
| add water to make | 1 | L |
| pH 7.0 | | |

### Example 7

In this example, a blood diluent is made by mixing the following into a final volume of 1 L:

| | | |
|---|---|---|
| theophylline | 0.5 | g |
| sodium sulfate | 10 | g |
| sodium chloride | 3.5 | g |
| phosphate buffer solution | 0.05 | M |
| add water to make | 1 | L |
| pH 7.0 | | |

### Comparative Example 2

The composition of the blood diluent is the same as that of Comparative Example 1.

### Example 8

In this example, a blood diluent is made by mixing the following into a final volume of 1 L:

| | | |
|---|---|---|
| uric acid | 0.5 | g |
| sodium sulfate | 10 | g |
| sodium chloride | 3.5 | g |
| phosphate buffer solution | 0.05 | M |
| add water to make | 1 | L |
| pH 7.0 | | |

### Comparative Example 3

The composition of the blood diluent is the same as that of Comparative Example 1.

### Example 9

In this example, a blood diluent is made by mixing the following into a final volume of 1 L:

| | | |
|---|---|---|
| caffeine | 0.5 | g |
| sodium sulfate | 10 | g |
| sodium chloride | 3.5 | g |
| phosphate buffer solution | 0.05 | M |
| add water to make | 1 | L |
| pH 7.0 | | |

### Comparative Example 4

The composition of the blood diluent is the same as that of Comparative Example 1.

### Example 10

In this example, a blood diluent is made by mixing the following into a final volume of 1 L:

| | | |
|---|---|---|
| hypoxanthine | 0.5 | g |
| sodium sulfate | 10 | g |
| sodium chloride | 3.5 | g |
| phosphate buffer solution | 0.05 | M |
| add water to make | 1 | L |
| pH 7.0 | | |

### Comparative Example 5

The composition of the blood diluent is the same as that of Comparative Example 1.

### Example 11

Each of the blood diluents obtained above is used in blood analysis. Mindray BC-3000 Plus blood cell analyzer (and BC-1800, BC-2800, BC-2300 blood cell subpopulations analyzers) are employed herein. Those skilled in the art will recognize that blood cell analyzers from other manufacturers or of other brands may also be adapted for use with the present disclosure.

During blood sample analysis, the numerous cells in the blood may overlap or clump together with each other potentially frustrating an accurate counting and volume measurement. As such, there exists a need to dilute the blood sample with a blood diluent so that the diluted blood cells may pass individually through a detection orifice of a blood cell analyzer. Moreover, a blood diluent may provide an ionic environment which may be advantageous for blood cell counting and the determination of cell number and volume.

One embodiment of the present disclosure may include a method wherein whole blood and the blood diluent can be mixed to form a diluted sample having a first concentration. Said diluted sample of the first concentration is divided into two portions. One portion is mixed with a certain amount of the blood diluent to form a diluted sample having a second concentration that is used for the counting of red blood cells and platelets. The other portion is mixed with a certain amount of a lysing agent to form a diluted sample of a third concentration that is used for the counting of white blood cells.

Another embodiment may include a pre-dilution method wherein there is an additional step called “dilution outside of the instrument”, namely, the capillary blood sample is mixed with a certain amount of the blood diluent outside the blood cell analyzer to form a diluted sample similar to the previously mentioned diluted sample of the first concentration. Following the pre-dilution method, sample analysis may proceed as disclosed for the whole blood method.

In one embodiment, the blood diluent of the present disclosure is adapted for all the dilution steps described herein, and is especially adapted for the dilution steps in the pre-dilution method, wherein a lysing agent may include the following formulation:

Lysing agent:

| | | |
|---|---|---|
| triazole | 10 | g |
| dodecyltrimethylammonium chloride (50% solution) | 35 | ml |
| tetradecyltrimethylammonium bromide | 3.5 | g |
| add water to make | 1 | L |
| pH 6.19 | | |

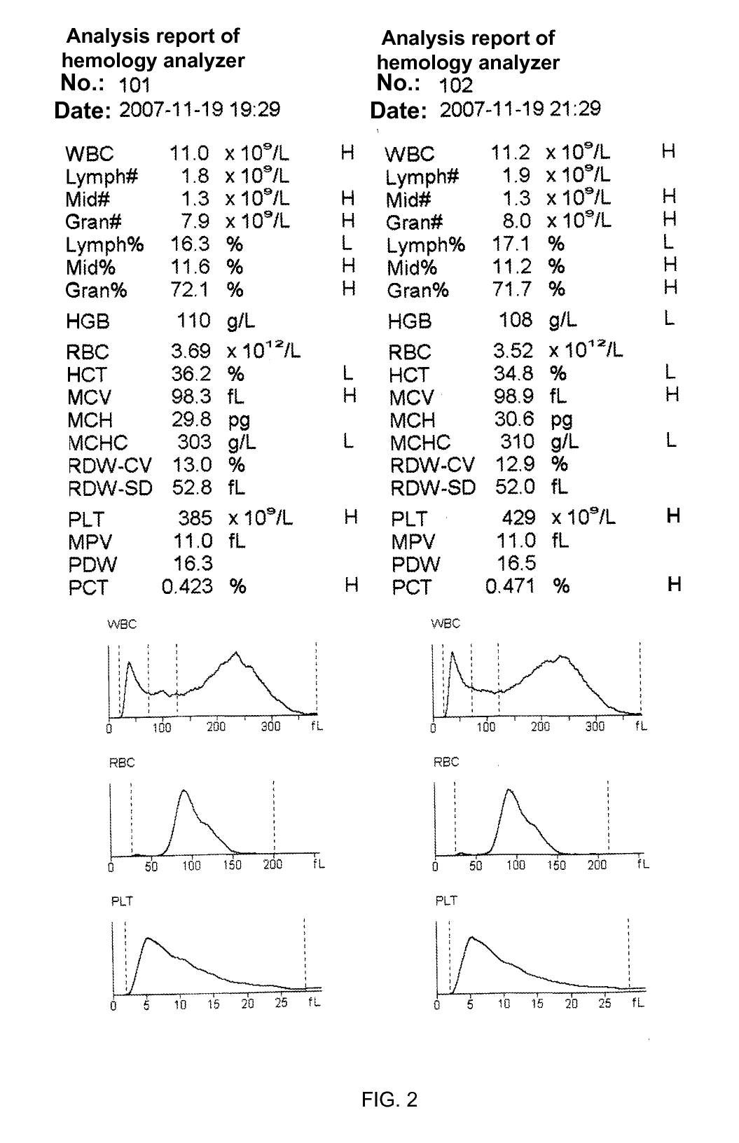

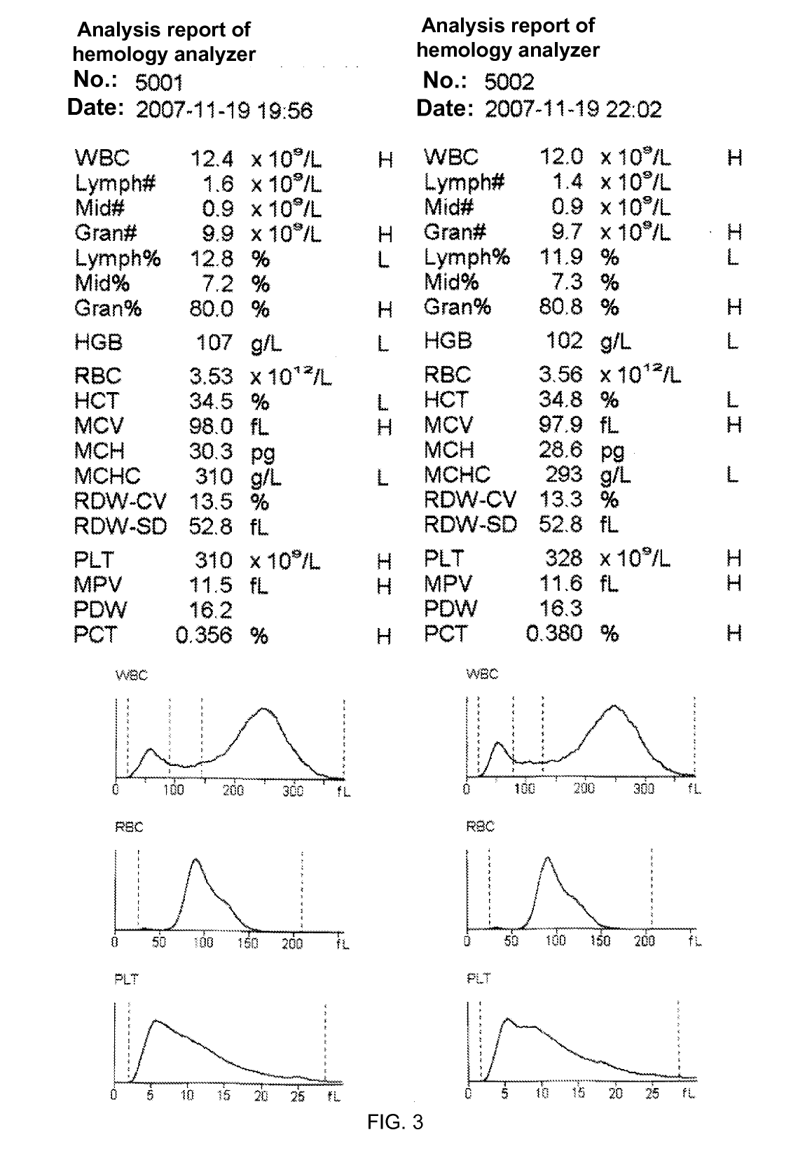

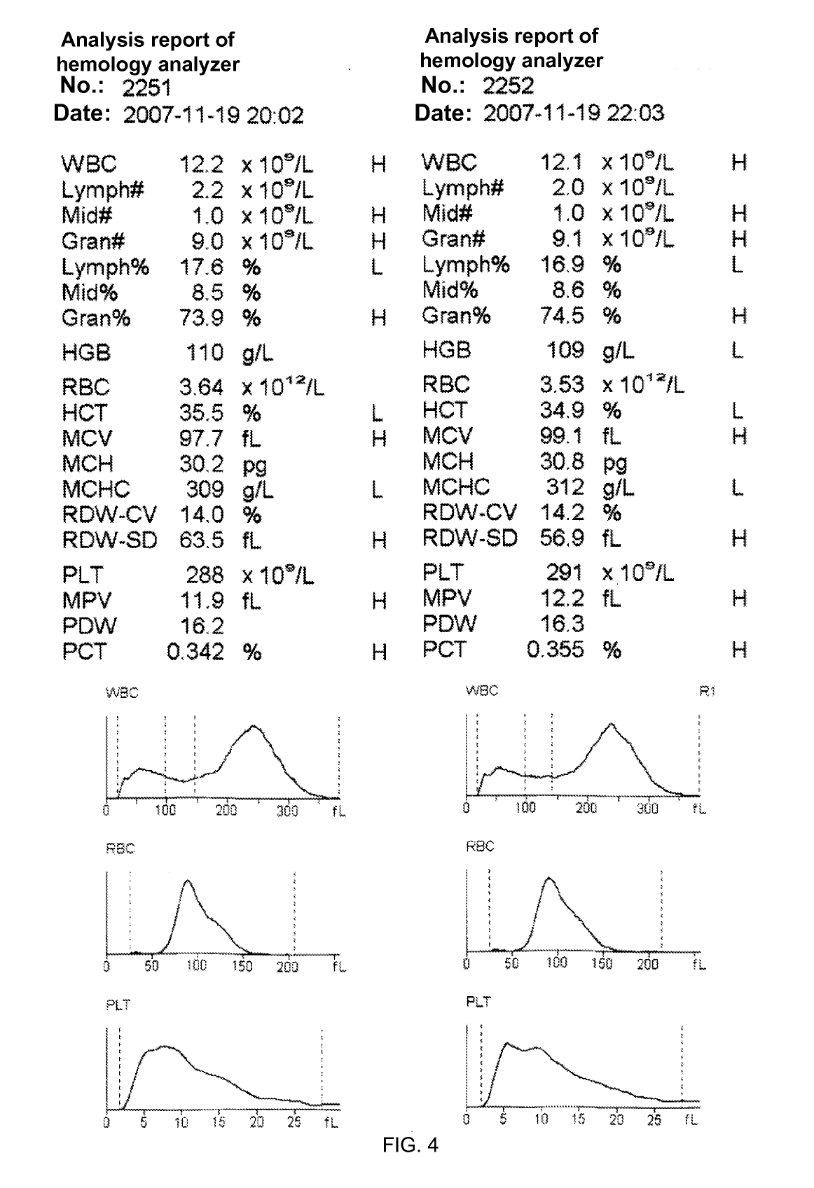

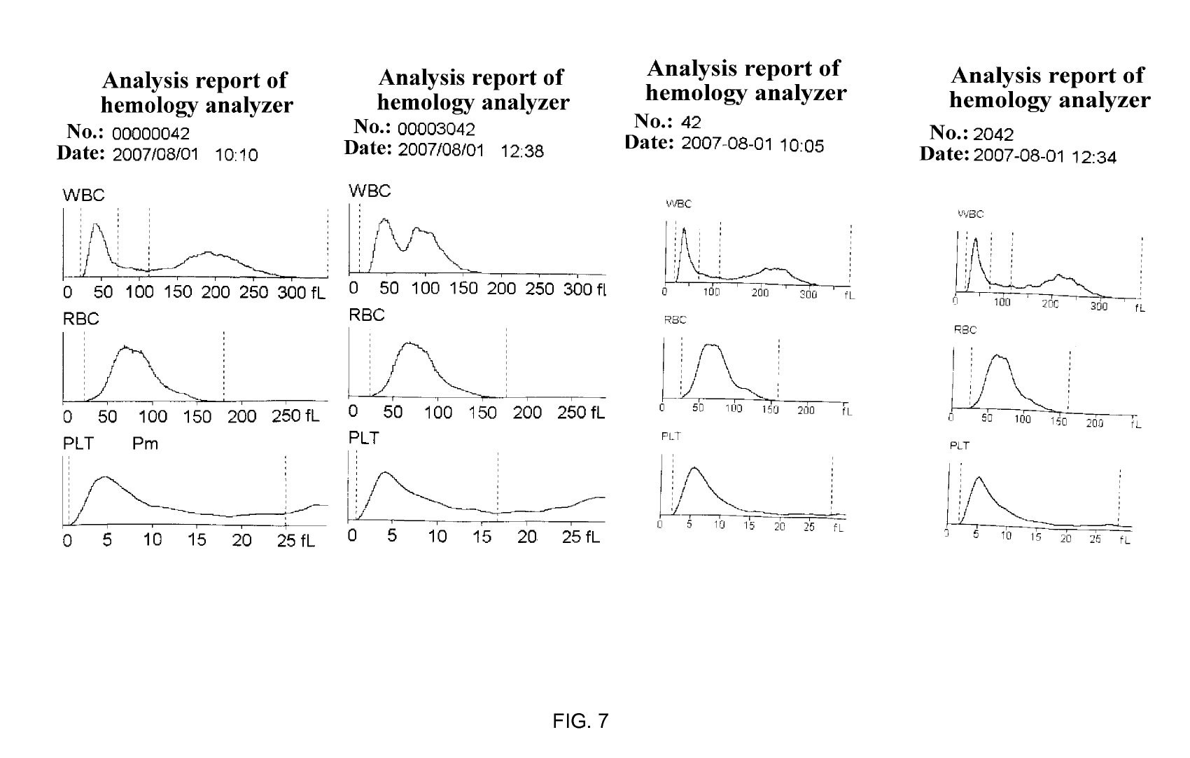

FIGS. 2-11 show the related histograms of white blood cells, red blood cells and platelets determined after the capillary blood samples are mixed with the blood diluents of the present disclosure. The samples as used are all collected from fresh clinical capillary blood samples. The left panels and the right panels in FIGS. 2-6 are the test results from the same blood sample, with the left panels obtained on fresh sample (about 5 minutes after sampling) and the right panels obtained on the same sample stored for at least 2 hours after sampling. Each of FIGS. 7-11 shows a comparison of two sets of histograms of the same blood sample obtained using two different blood diluents according to the present disclosure (see the description of the titles of each figure). In each of the histograms, the left panels are obtained on fresh sample (about 5 minutes after sampling, not more than 10 minutes) and the right panels are obtained on the same sample stored for at least 2 hours after sampling.

FIGS. 2-4, respectively, correspond to examples 1-3 wherein the diluent contains caffeine in a concentration of 0.05 g/L, 0.5 g/L and 25 g/L, respectively. It can be seen from FIGS. 2-4 that the curves of the blood components (white blood cells, red blood cells, platelets) obtained after the blood sample was diluted with a blood diluent according to the present disclosure and stored for 2 hours, are substantially the same as those curves obtained from a sample analyzed only 5-minutes after collection.

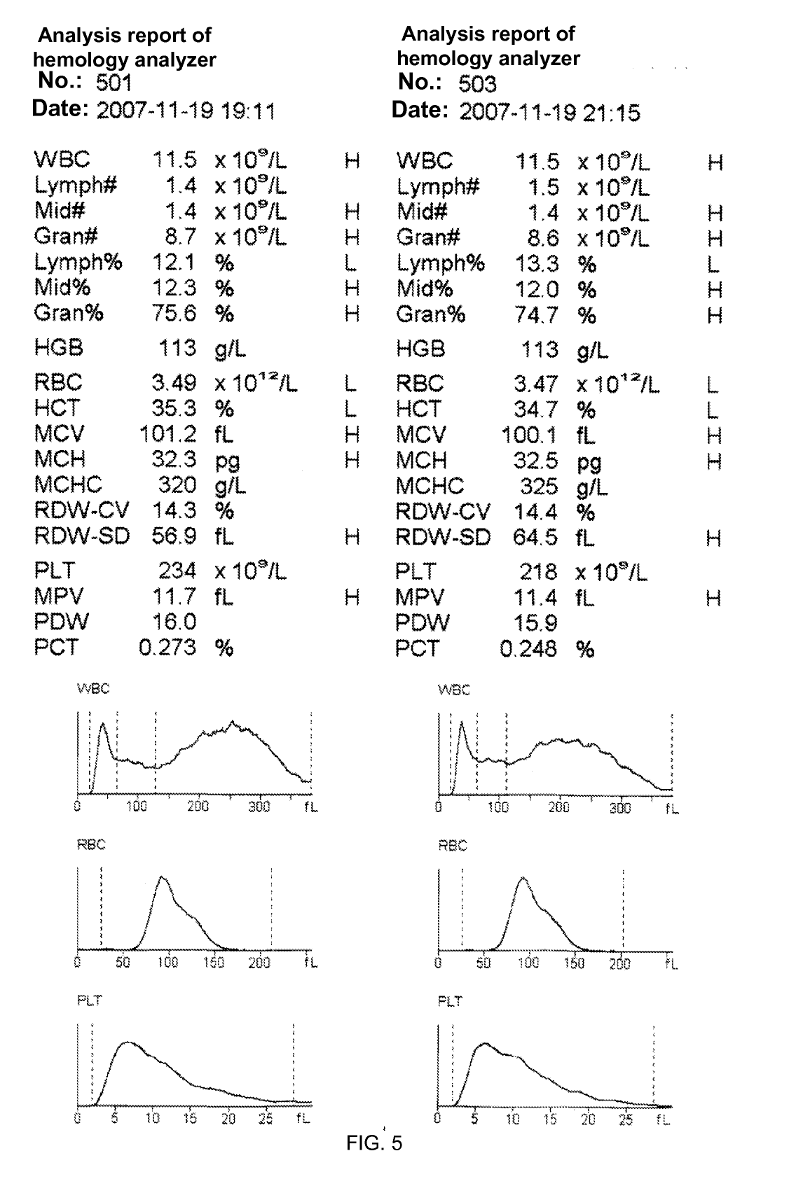

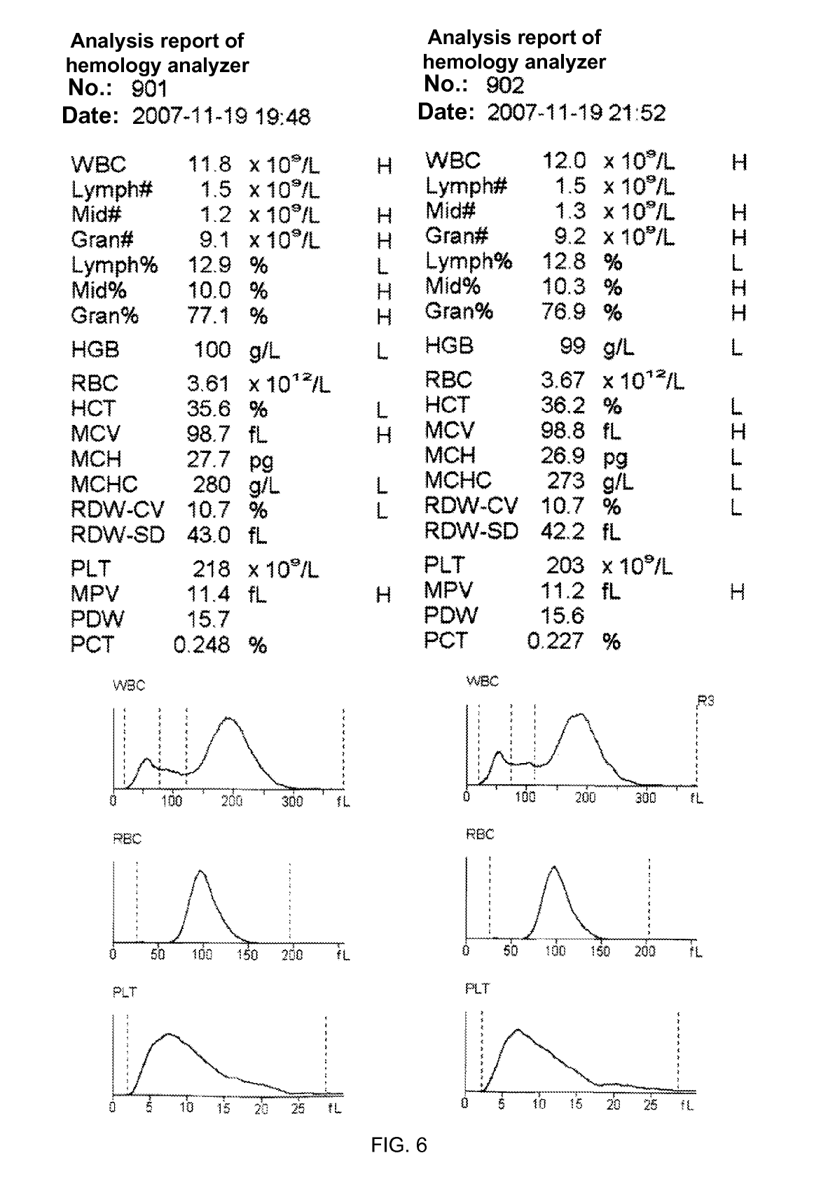

FIGS. 5 and 6 correspond to examples 4 and 5, respectively, wherein caffeine is used and the pH values of the blood diluent as used are pH 5.0 and pH 9.5 respectively. It can be seen from FIGS. 5 and 6 that blood diluents prepared according to the present disclosure with a pH of approximately 5.0 and 9.5 can both effectively stabilize the blood samples beyond 2 hours after collection.

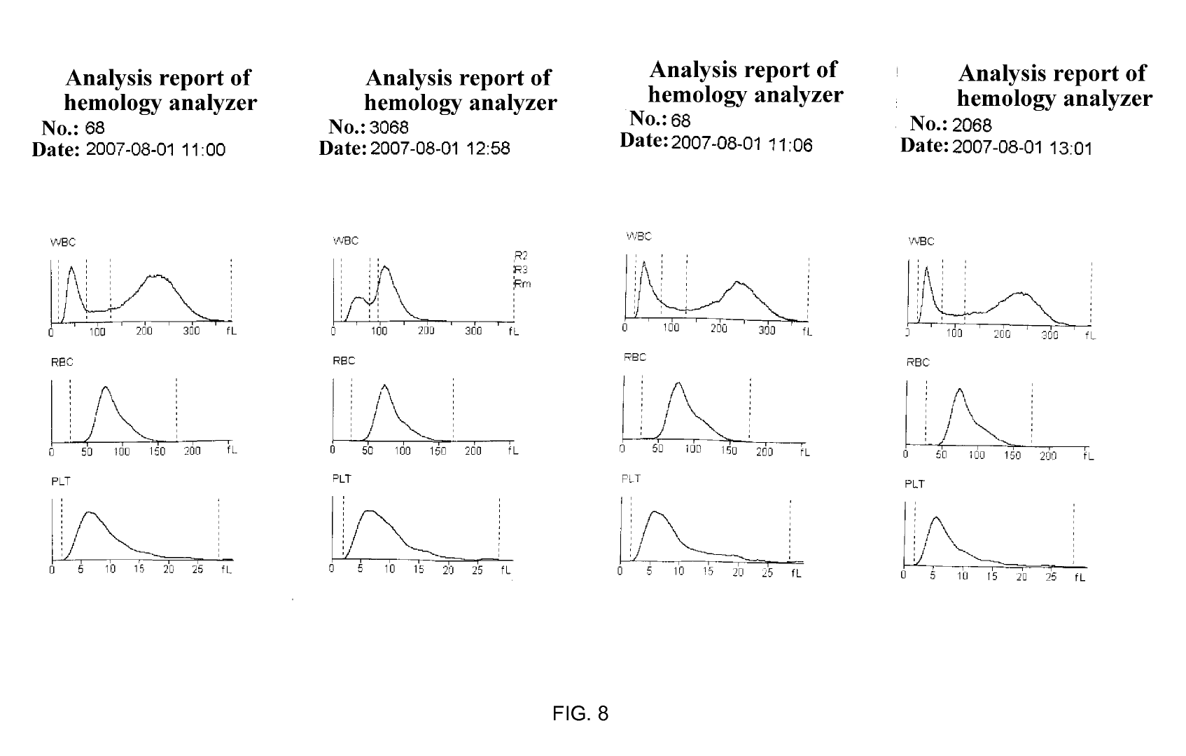

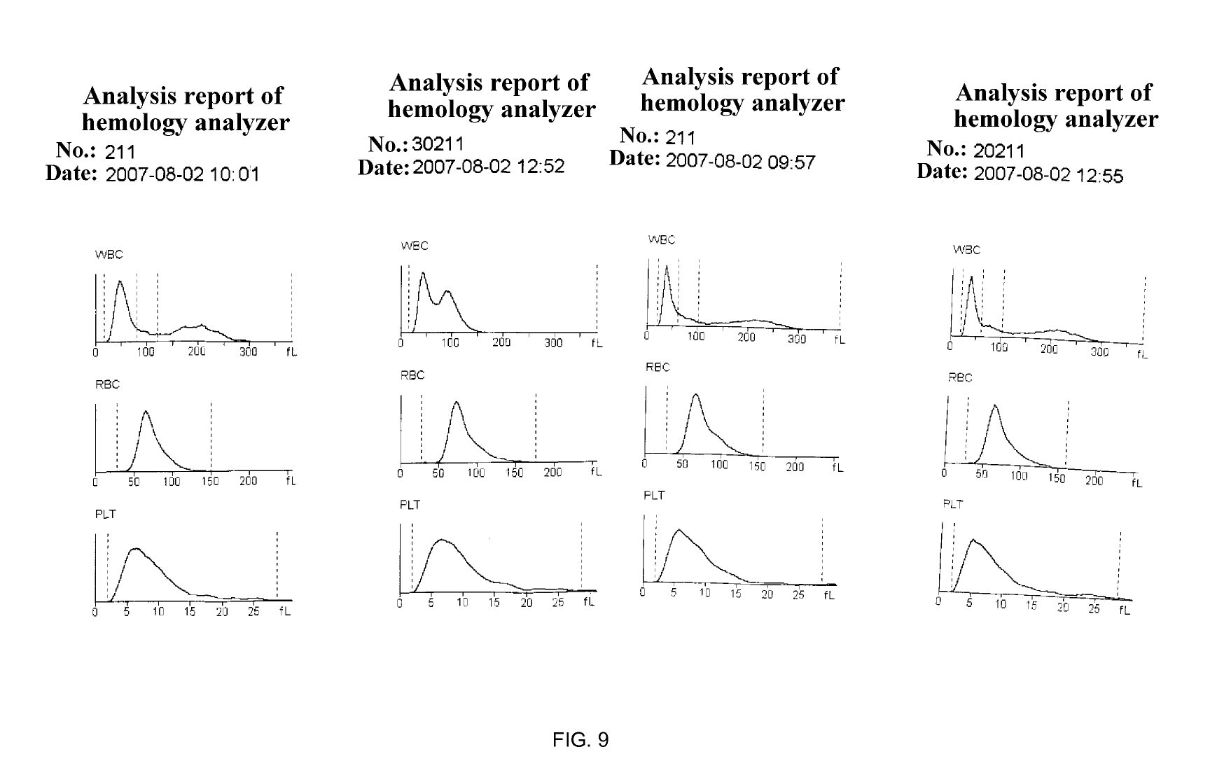

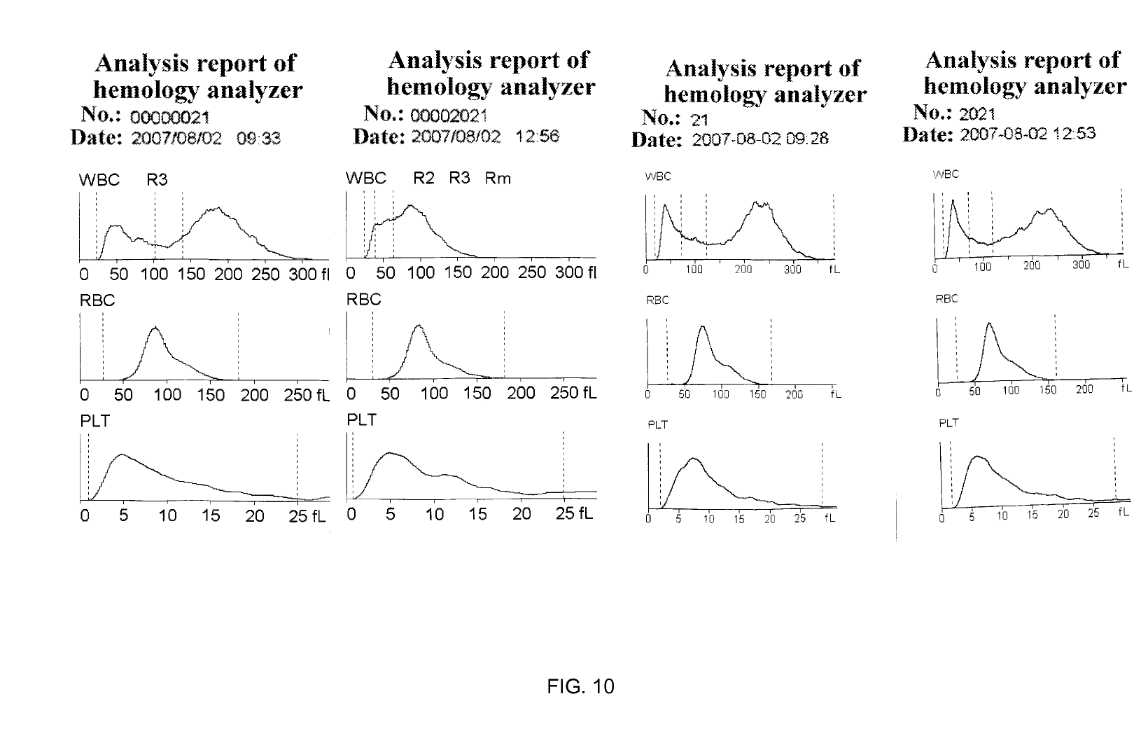

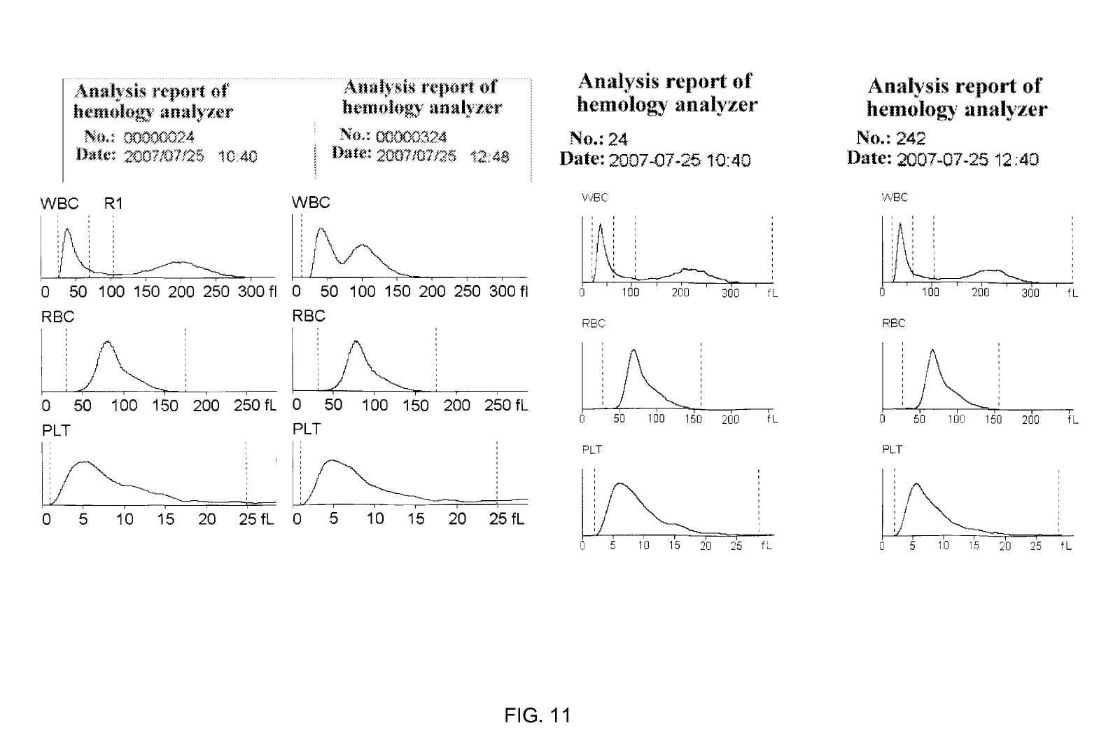

FIGS. 7-11 correspond to examples 6-10, respectively, wherein aminophylline, theophylline, uric acid, caffeine and hypoxanthine are used, and to comparative examples 1-5 wherein N-(2-acetamido)-2-iminodiacetic acid (ADA), dimethylurea and (2-pyridinethiol-1-oxide) sodium are used. Examples 6-10 and comparative examples 1-5 are used as stability tests of a blood diluent prepared according to the present disclosure. It can be seen from the figures that the blood diluents of examples 6-10 can effectively stabilize the blood components in the blood sample for a period of time (FIG. 7, 2 hours and 33 minutes; FIG. 8, 2 hours and 3 minutes; FIG. 9, 3 hours; FIG. 10, 3 hours and 30 minutes and FIG. 11, 2 hours and 5 minutes), In contrast, the histograms of comparative examples 1-5 for white blood cells change considerably during the same time.

It is understood from the above specific embodiments that the various embodiments of blood diluents according to the present disclosure can effectively stabilize red blood cells, white blood cells in the blood sample for at least 2, giving satisfactory results on a blood cell analyzers.

It will be understood by those having skill in the art that many changes may be made to the details of the above-described embodiments without departing from the underlying principles of the invention. The scope of the present invention should, therefore, be determined only by the following claims.

What is claimed is:

1. A blood diluent for use in the analysis of blood components, said blood diluent comprising:
at least one purine compound or salt thereof.

2. The blood diluent according to claim 1, said blood diluent further comprising:
at least one alkali metal salt; at least one organic buffer, and/or at least one inorganic buffer.

3. The blood diluent according to claim 1, wherein said at least one purine compound or salt thereof includes at least one hypoxanthine compound or salt thereof or at least one xanthine compound or salt thereof.

4. The blood diluent according to claim 3, wherein said at least one hypoxanthine compound or salt thereof or at least one xanthine compound or salt thereof include substances selected from the group consisting of: caffeine, theophylline, aminophylline, choline theophyllinate, diprophylline, doxofylline, cafedrine, bamifylline, theobromine, pentoxifylline, propentofylline, denbufyline, hypoxanthine, xanthine, 1-methylxanthine, 3-methylxanthine, 7-methylxanthine, 1,7-dimethylxanthine, uric acid, 1-methyluric acid, 1,3-dimethyluric acid, 7-methyluric acid and combinations, derivatives and salts thereof.

5. The blood diluent according to claim 1, wherein said at least one purine compound or salt thereof are in a concentration ranging from approximately 0.05 g/L to approximately 25 g/L.

6. The blood diluent according to claim 1, wherein said at least one purine compound or salt thereof are in a concentration ranging from approximately 0.3 g/L to approximately 10 g/L.

7. The blood diluent according to claim 1, wherein said at least one purine compound or salt thereof are in a concentration of approximately 0.5 g/L.

8. The blood diluent according to claim 2, wherein said at least one alkali metal salt is selected from alkali metal sulfates and alkali metal halides.

9. The blood diluent according to claim 2, wherein said blood diluent has a conductivity ranging from approximately 16 mS/cm to approximately 22 mS/cm and an osmotic pressure ranging from approximately 250 mOsm/Kg to approximately 350 mOsm/Kg.

10. The blood diluent according to claim 2, wherein said at least one organic buffers and/or at least one inorganic buffers are selected from the group consisting of formic acid, phthalic acid, acetic acid, phosphoric acid, TRIS, boric acid, carbonic acid and salts thereof and combinations thereof.

11. The blood diluent according to claim 2, wherein said blood diluent has a pH value ranging from approximately 5.0 to approximately 9.5.

12. The blood diluent according to claim 2, wherein said blood components are selected from red blood cells, white blood cells, platelets and hemoglobin.

13. A method for analyzing blood components, said method comprising the step of mixing the blood sample with the blood diluent of claim 1 and at least one lysing agent.

14. A method for analyzing blood components, said method comprising the step of mixing the blood sample with the blood diluent of claim 2 and at least one lysing agent.
